(19) United States
(12) Patent Application Publication
(10) Pub. No.: US 2026/0207342 A1
(43) Pub. Date: Jul. 23, 2026

(54) TOTAL ANKLE REPLACEMENT WITH GRADIENT POROUS STRUCTURE

(71) Applicant: Enovis Corporation, Lewisville, TX (US)

(72) Inventor: Samuel Fang, Plano, TX (US)

(21) Appl. No.: 19/450,936

(22) Filed: Jan. 16, 2026

Related U.S. Application Data
(60) Provisional application No. 63/746,401, filed on Jan. 17, 2025.

(51) Int. Cl. A61F 2/42; A61F 2/30; A61L 27/06; A61L 27/56; B33Y 80/00

(52) U.S. Cl. CPC A61F 2/4202; A61F 2/30771; A61F 2/3094; A61L 27/06; A61L 27/56; B33Y 80/00; A61F 2002/30011; A61F 2002/3092; A61F 2002/3093; A61F 2002/30985; A61F 2002/4205; A61F 2002/4207; A61F 2310/00023

(57) ABSTRACT

A gradient porous structure for one or more bone contacting surfaces of a total ankle replacement implant is provided. The gradient porosity of the porous structure may be optimized to match the natural variation of a patient's bone density, while the pattern of the porous structure may be optimized to maximize mechanical strength so that the implant is structurally stable within the ankle joint.

### CROSS REFERENCE TO RELATED APPLICATIONS

This application claims priority to and the benefit of U.S. Provisional Patent Application No. 63/746,401, filed on Jan. 17, 2025, which is hereby incorporated by reference in its entirety.

### FIELD

This disclosure relates to a total ankle replacement device. More specifically, this disclosure relates to a gradient porous structure for bone-contacting surfaces of a total ankle replacement device.

### BACKGROUND

Total ankle replacement is a procedure that is used for patients with, for example, osteoarthritis, post-traumatic arthritis, or rheumatoid arthritis affecting the ankle joint. Metallic porous structures are commonly used on bone contacting surfaces of total ankle replacement implants, among others, to promote bone ingrowth to provide the total ankle replacement implant with better stability because the porous structures have a higher coefficient of friction and facilitate osteointegration with the surface morphology of the bone.

Existing porous structures that are commonly used in orthopedic implants, such as total ankle replacement implants, have a uniform structure and porosity. However, the bones to which these implants are attached do not have uniform densities. In the context of total ankle replacement implants, for example, the tibia and talus have low density cancellous bone towards the center of the bone, with high density cortical bone on the outer edges thereof. So, the porosity of the bone contacting surfaces of the total ankle replacement implants does not match the porosity of the bone being contacted by the porous surface, making bone ingrowth more difficult.

It is therefore desirable to provide total ankle replacement implants with porous bone contacting surfaces that mimic the natural bone porosity to improve bone ingrowth with said implants, thereby improving the strength and stability thereof.

### SUMMARY

In one aspect, an orthopedic implant is provided. The orthopedic implant may include a bone contacting surface, where the bone contacting surface has a gradient porous structure to facilitate bone ingrowth within the bone contacting surface. The gradient porous structure may have a porous pattern. In some aspects, the porous pattern is uniform across the bone contacting surface. In some aspects, the porous pattern has a variable porosity across the bone contacting surface. In some aspects, the porous pattern has a first porosity at a center of the bone contacting surface and a second porosity at a perimeter of the bone contacting surface, wherein the first porosity is greater than the second porosity. The porosity of the porous pattern may linearly decrease from the first porosity at the center of the bone contacting surface to the second porosity at the perimeter of the bone contacting surface. The first porosity may be from about 80% to about 90%, preferably about 82%, and the second porosity may be from about 40% to about 55%, preferably about 52%. In some aspects, a pore size of the porous pattern is from about 200 μm to about 600 μm. In some aspects, an average pore size of the porous pattern is about 450 μm. In some aspects, the porous pattern is formed according to a triply periodic minimal surface (TPMS) function.

In another aspect, a total ankle replacement implant is provided. The total ankle replacement implant may include a stem, a tibial tray, and a talus component, where each of the stem, the tibial tray, and the talus component have at least one bone contacting surface having a gradient porous structure to facilitate bone ingrowth within the at least one bone contacting surface. The gradient porous structure may have a porous pattern. In some aspects, the gradient porous structure of the one or more bone contacting surfaces of each of the stem, the tibial tray, and the talus component is 3D printed titanium.

In some aspects, the porous pattern is formed according to a triply periodic minimal surface (TPMS) function. In some aspects, the porous pattern has a first porosity at a center of each bone contacting surface and a second porosity at a perimeter of each bone contacting surface, wherein the first porosity is greater than the second porosity. The density of the porous pattern may linearly decrease from the first porosity at the center of each bone contacting surface to the second porosity at the perimeter of each bone contacting surface. The first porosity may be from about 80% to about 90%, preferably about 82%, and the second porosity may be from about 40% to about 55%, preferably about 52%. In some aspects, a pore size of the porous pattern is from about 200 μm to about 600 μm, and wherein an average pore size of the porous pattern is about 450 μm.

In a further aspect, a method for inserting a total ankle replacement implant in an ankle joint of a patient is provided. The method may include the steps of resecting a tibia or a talus within the ankle joint of the patient, and inserting a component of the total ankle replacement implant into the ankle joint of the patient, the component of the total ankle replacement implant being configured to be attached to the tibia or the talus after resection. The component of the total ankle replacement implant may have a bone contacting surface with a gradient porous structure to facilitate bone ingrowth within the bone contacting surface and the tibia or the talus. The gradient porous structure may include a porous pattern having a first porosity at a center of the bone contacting surface and a second porosity at a perimeter of the bone contacting surface, where the first porosity is greater than the second porosity. In some aspects, the first porosity is from about 80% to about 90%, preferably about 82%, and the second porosity is from about 40% to about 55%, preferably about 52%.

### BRIEF DESCRIPTION OF THE DRAWINGS

The detailed description is set forth with reference to the accompanying drawings. The use of the same reference numerals may indicate similar or identical items. Various embodiments may utilize elements and/or components other than those illustrated in the drawings, and some elements and/or components may not be present in various embodiments. Elements and/or components in the figures are not necessarily drawn to scale.

FIG. 1 is a perspective view of a total ankle replacement implant, according to one or more embodiments of the present disclosure.

FIG. 2 is a perspective view of a stem component of the total ankle replacement implant of FIG. 1, according to one or more embodiments of the present disclosure.

FIG. 3 is a perspective view of a tibial tray component of the total ankle replacement implant of FIG. 1, according to one or more embodiments of the present disclosure.

FIG. 4. is a perspective view of a talus component of the total ankle replacement implant of FIG. 1, according to one or more embodiments of the present disclosure.

FIG. 5A depicts a gradient porous structure pattern for implementation on a bone-contacting surface of the total ankle replacement implant of FIG. 1, according to one or more embodiments of the present disclosure.

FIG. 5B is a magnified view of the gradient porous structure pattern of FIG. 5A, according to one or more embodiments of the present disclosure.

FIG. 6 depicts experimental gradient porous structures, according to one or more embodiments of the present disclosure.

FIGS. 7A-7B are graphs of the static (FIG. 7A) and kinetic (FIG. 7B) coefficients of friction for the gradient porous structure patterns of FIG. 6, according to one or more embodiments of the present disclosure.

FIGS. 8A-8B are graphs of the static (FIG. 8A) tensile stress and the shear (FIG. 8B) tensile stress for the two gradient porous structure patterns of FIG. 6 having the greatest coefficients of friction, according to one or more embodiments of the present disclosure.

FIG. 9 is a graph of the abrasion mass loss for the two gradient porous structure patterns of FIG. 6 having the greatest coefficients of friction, according to one or more embodiments of the present disclosure.

### DETAILED DESCRIPTION

The disclosure will be described more fully hereinafter with reference to the accompanying drawings, in which example embodiments of the disclosure are shown. This disclosure may, however, be embodied in many different forms and should not be limited to the embodiments set forth herein.

Gradient porous structures as described herein may be implemented on one or more bone contacting surfaces of a total ankle replacement implant. The gradient porosity of the porous structure may be optimized to match the natural variation of a patient's natural anatomical bone density, while the pattern of the porous structure may be optimized to maximize mechanical strength (i.e., to maximize the coefficient of friction) so that the implant is structurally stable. In some embodiments, the gradient porous structures may also be etched to further promote bone ingrowth and cell differentiation and proliferation within the bone ingrowth.

A total ankle replacement implant is provided, having such a gradient porous structure on its bone contacting surfaces. The gradient porous structure may address limitations in current ankle replacement implants, which typically use uniform porous structures that do not match the natural variation in a patient's bone density, where the cancellous bone at the center has a low density and the cortical bone around the edges has a high density. By mimicking this natural gradient, the total ankle replacement implant described herein aims to improve bone ingrowth and osteointegration, thereby enhancing stability and longevity of said implant.

The total ankle replacement implant may include a stem, tibial tray, articulating surface component, and talus component, with the stem, tibial tray, and talus component each being made of a metallic material. Each of these metallic components—typically made from titanium or cobalt-chromium alloys—is designed with gradient porous bone contacting surfaces to facilitate bone ingrowth. The gradient porous structure is achieved through 3D printing and may include additional nanoscale etching to promote cell proliferation and differentiation. The porosity varies from about 80-90% at the center to about 45-55% at the edges, with pore sizes ranging from 200 μm to 600 μm, averaging around 450 μm. The porous pattern may be based on a triply periodic minimal surface (TPMS) function, which optimizes mechanical strength and frictional properties.

In certain embodiments, a total ankle replacement having one or more bone contacting surfaces is provided. In some embodiments, the one or more bone contacting surfaces are present on the stem, tibial tray, and talar components of the total replacement implant. For example, an outer surface of the stem, a top surface and/or one or more side surfaces of the tibial tray, and a bottom surface of the talar component may have a gradient porous structure to facilitate bone ingrowth. The gradient porous structure may have a porous pattern that supports the mechanical strength of the implant, with the porous pattern being consistent across the bone contacting surface (i.e., the shape of the pores is the same throughout the entire pattern, across the entire bone contacting surface). The porous pattern may also have a variable porosity across the bone contacting surface, in that each bone contacting surface may have a high porosity (i.e., large pores) at the center of the surface, and the porosity (i.e., size of the pores) gradually decreases towards the perimeter of the surface to a low porosity (i.e., small pores). The variable porosity of the gradient porous structure may be similar to the density of a patient's natural bone to which the bone contacting surface of the implant component is attached.

### Total Ankle Replacement Implant

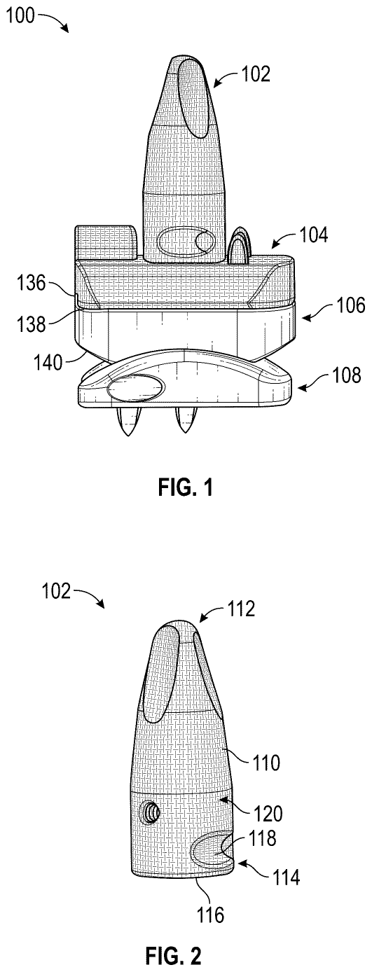

A total ankle replacement implant, which may have one or more bone contacting surfaces having a gradient porous structure as described herein, is shown in FIG. 1. In embodiments, the total ankle replacement implant 100 includes the following components: a stem 102, a tibial tray 104, an articulating surface component 106, and a talus component 108. Each of the stem 102, tibial tray 104, and talus component 108 may be fabricated from a metallic material, such as titanium, a titanium alloy, cobalt chromium, a cobalt chromium alloy, or a combination thereof, and may have one or more bone contacting surfaces having a gradient porous structure as described herein. In some embodiments, the stem 102, tibial tray 104, and talus component 108 are manufactured using 3D printing or additive manufacturing methods, so that the stem 102, tibial tray 104, and talus component 108 are each solid while still having one or more bone contacting surfaces having a gradient porous structure to facilitate bone ingrowth and promote osteointegration. In some embodiments, the articulating surface component 106 is formed of a biocompatible polymer, such as ultra-high-molecular-weight polyethylene (UHMWPE), cross-linked UHMWPE, polyether ether ketone (PEEK), or a combination thereof.

In embodiments, the stem 102, tibial tray 104, articulating surface component 106, and talus component 108 are assembled to form the total replacement implant 100 as shown in FIG. 1. When the total ankle replacement implant 100 is assembled within a patient's ankle joint, the stem 102 is placed within a canal resected within the patient's tibia, with the tibial tray 104 being attached to the stem 102 and the resected tibia at a distal end thereof. The articulating surface component 106 attaches to the distal end of the tibial tray 104, and is movably engaged with the talus component 108, which is fixed to the proximal end of the patient's resected talus bone. The total ankle replacement implant 100 may therefore have one or more bone contacting surfaces, each of which may be formed of a porous structure to facilitate bone ingrowth. According to preferred embodiments, the one or more bone contacting surfaces have the gradient porous structure described herein.

In preferred embodiments, the stem 102, tibial tray 104, and talus component 108, each having one or more bone contacting surfaces, are shown and described in greater detail with respect to FIGS. 2-4.

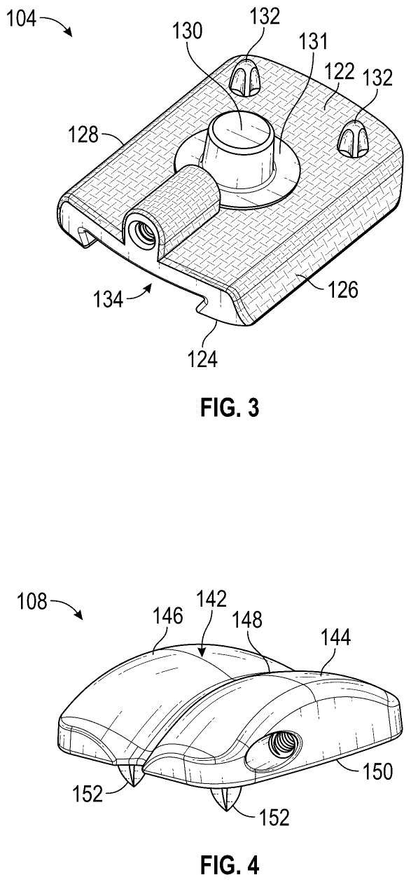

In embodiments, as shown in FIG. 2, the stem 102 includes an elongated body 110 defining a first (or proximal) end 112 and a second (or distal) end 114. In some embodiments, the elongated body 110 is generally cylindrical at the distal end 114 thereof, and tapers in a direction towards the proximal end thereof 112. In other embodiments (not shown), the elongated body 110 may be fully cylindrical. The proximal end 114 also defines a bottom surface 116 that interfaces with the tibial tray 104. In some embodiments, the bottom surface 116 defines a cavity therein (not shown) for receiving a reciprocal protrusion of the tibial tray 104, as shown and described in greater detail with respect to FIG. 3, for securing the stem 102 and the tibial tray 104 together. In some embodiments, the proximal end 114 of the elongated body 110 of the stem 102 may also include one or more indents 118, which may be configured to receive a fixture therein, the fixture being used to aid gripping and holding of the stem 102 during insertion into the patient's ankle joint. In use, the stem 102 may be inserted into the patient's tibial canal such that the entire outer surface 120 of the elongated body 110 contacts the interior of the patient's tibia. The outer surface 120 may therefore have a gradient porous structure as described herein to facilitate bone ingrowth.

While the stem 102 is shown and described herein as having a single unitary elongated body 110, it would be understood that the stem 102 may have any shape, size, and/or structure suitable for insertion into the tibial canal during a total ankle replacement procedure, as would be understood by those skilled in the art. For example, the stem 102 may be segmented or flexible to facilitate insertion into the tibial canal, as described in PCT/US2025/043710, which is incorporated by reference herein in its entirety.

When the total ankle replacement implant 100 is assembled, the stem 102 is attached to and extends proximally from the tibial tray 104. In embodiments, the tibial tray 104, as shown in FIG. 3 includes a top (or proximal) surface 122 that contacts and engages with the patient's tibia, and a bottom (or distal) surface 124 that is configured to engage with the articulating surface 106 component of the implant. In some embodiments, the tibial tray 104 may also have one or more side surfaces, such as the medial side surface 126 and the lateral side surface 128, that also at least partially contact the tibia. The top surface 122, medial side surface 126, and/or lateral side surface 128, if bone contacting, may have a gradient porous structure as described herein to facilitate bone ingrowth.

The tibial tray 104 may also include a projection 130 extending from the top surface 122 of the tray 104 that is configured to engage with the stem 102. Specifically, the projection 130 is configured to be received within the cavity (not shown) defined in the bottom surface 116 of the elongated body 110 of the stem 102 for securing the stem 102 and the tibial tray 104 together via, for example, an interference or press fit between the cavity (not shown) of the stem 102 and the projection 130 of the tibia tray 104. The projection 130 and the portion 131 of the top surface 122 adjacent to or surrounding the projection 130 may contact the stem 102 rather than the patient's bone, in which case the projection 130 and portion 131 of the top surface may not have the gradient porous structure and instead may be solid metal (i.e., titanium). In embodiments, the tibial tray 104 may also include one or more anchors 132 extending from the top surface 122 thereof to further secure the total ankle replacement implant 100 to the patient's tibia. While the anchors 132 are shown in FIG. 3 as not having a gradient porous structure, it would be understood that the anchors 132 may be fabricated with the gradient porous structure to promote ingrowth because the anchors 132 are bone contacting.

When the tibial tray 104 is assembled with the rest of the total replacement implant 100, the tray 104 sits immediately below the patient's tibia, with the top surface 122 of the tray 104 being at least partially in contact therewithin. The bottom surface 124 of the tray 104 is configured to engage with the articulating surface 106 component of the implant 100, which is located in the gap between the patient's tibia and talus. In some embodiments, the bottom surface 124 of the tray 104 includes a slot 134 for receiving a projection 136 extending from a top side 138 of the articulating surface component 106, as shown in FIG. 1. The opposed bottom side 140 of the articulating surface component 106 defines a contoured surface (not shown) configured to engage and articulate about the talus component 108, mimicking the natural articulation of the ankle.

As shown in FIG. 4, the talus component 108 includes an upper articulating surface 142 having a medial condylar surface 144, a lateral condylar surface 146, and a notch 148 disposed therebetween. The upper articulating surface 142 of the talus component 108 engages with the bottom side 140 of the articulating surface component 106 of the implant 100. The size and shape of the talus component 108 may be tailored to a patient's anatomy, and is configured to interface with the bottom side 140 of the articulating surface component 106.

In embodiments, the talus component 108 includes a bottom surface 150 opposite the upper articulating surface 142. The bottom surface 150 may be substantially flat, and interfaces with the patient's talus to attach the total ankle replacement implant 100 thereto. The bottom surface 150 may be bone contacting and therefore may have a gradient porous structure as described herein to facilitate bone ingrowth. In some embodiments, the bottom surface 150 of the talus component 108 may also have one or more anchors 152 projecting therefrom to further secure the total ankle replacement implant 100 to the patient's talus. While the anchors 152 are shown in FIG. 4 as not having a gradient porous structure, it would be understood that the anchors 152 may be fabricated with the gradient porous structure to promote bone ingrowth because the anchors 152 are bone contacting.

### Gradient Porous Structures

One or more components of the total ankle replacement implant described herein may have at least one bone contacting surface having a gradient porous structure to facilitate bone ingrowth and osteointegration of the implant component with the patient's bone to which the component of the implant is attached. In embodiments, the implant components having the bone contacting surfaces are 3D printed, or fabricated using other additive manufacturing techniques, such that the body of each implant component is solid, and the outer most layer on the respective bone contacting surfaces have a gradient porous structure as described herein. In embodiments, these implant components and the gradient porous structure are fabricated from metallic materials such as titanium, a titanium alloy, cobalt chromium, a cobalt chromium alloy, or a combination thereof. In preferred embodiments, the gradient porous structures described herein are formed from 3D printed titanium.

The gradient porous structure on the one or more bone contacting surfaces of the total ankle replacement implant may have a macroscale level design, a microscale level design, and a nanoscale to facilitate integration of the gradient porous structure with the host tissue (i.e., the patient's natural bone). For example, the variable or gradient porosity of the gradient porous structure (i.e., the macroscale level design) may mimic natural bone density variation, while the pattern of the gradient porous structure (i.e., the microscale level design) may provide an interconnected porous network to mimic bone morphology and mechanical properties to facilitate bone regrowth. In some embodiments, etching within the gradient porous structure (i.e., the nanoscale level design) may be provided to promote cell proliferation and differentiation within the bone regrowth. In embodiments, the variable or gradient porosity and the pattern of the gradient porous structure are achieved through the 3D printing of the structure. In embodiments, the etching within the structure may be achieved by chemical (i.e., acid) or laser etching.

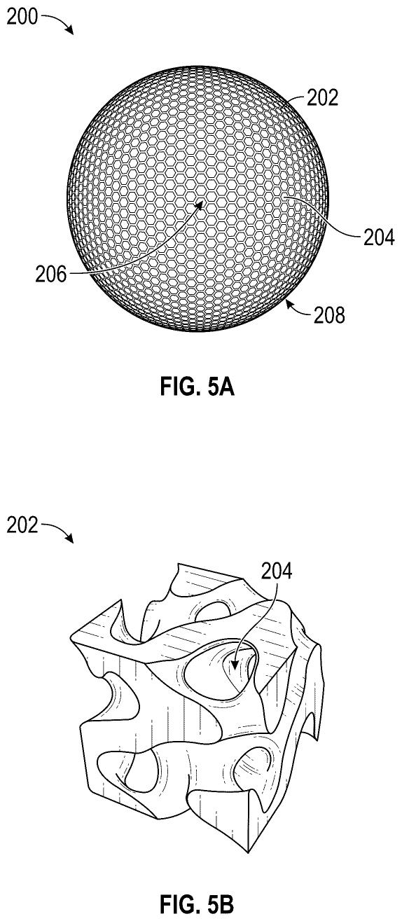

In embodiments, a uniform layer of the gradient porous structure is provided on one or more bone contacting surfaces of the total ankle replacement implant, as described with respect to FIGS. 1-4. As shown in FIG. 5A, the gradient porous structure 200 may have a porous pattern 202 that is consistently shaped across the entire porous surface 200. The pattern 202 may define a plurality of pores 204, each having the same or different shapes, and ranging in size from about 200 μm to about 600 μm, with an average pore size of about 450 μm, across the largest dimension.

While the pattern 202 of the gradient porous structure 200 is consistent across the entire bone contacting surface, the porosity of the pattern 202 and size of the pores 204 varies across the surface to mimic the natural variation in bone density. For example, the porosity of the pattern 202 is highest (i.e., the size of the pores 204 are the largest) at a center 206 of the bone contacting surface, while the porosity of the pattern 202 is the lowest (i.e., the size of the pores 204 are the smallest) along a periphery 208 of the bone contacting surface. This mirrors the natural variation in bone density, where the density of the cancellous bone at the center of the bone is the lowest, and the density increases towards the outer cortical ring at the edge of the bone, where the density is the highest. In embodiments, the porosity of the gradient porous structure 200 ranges from between about 45% and about 90%, depending on the surface location. According to preferred embodiments, the porosity of the gradient porous structure is between about 52% and about 82%.

In embodiments, the gradient porous structure 200 of the stem component of the total ankle replacement implant has a porosity that is similar to the porosity at the center of the tibia. In some embodiments, the entire gradient porous structure 200 of the stem component has a porosity of between about 80% to about 90%, preferably about 82%. In other embodiments, the gradient porous structure 200 of the stem component has a porosity of between about 80% to about 90%, preferably about 82%, at the tip thereof, and the porosity will decrease slightly down the length of the stem as it tapers outward relative to the tip thereof. In some embodiments, the porosity linearly decreases down the length of the stem.

In embodiments, the gradient porous structure 200 of the bone contacting surface of the tibial tray of the total replacement implant, i.e., the top surface of the tibial tray, has a porosity that mimics the porosity of the tibia. For example, the porosity of the gradient porous structure 200 of the top side of the tibial tray, contacting the tibia, has a porosity between about 80% to about 90% at the center, and a porosity of about 45% to about 55% around the edges. According to preferred embodiments, the porosity at the center of the tibial tray is about 82%, and the porosity around the edges of the tibial tray is about 52%. The porosity may decrease linearly from the center of the tibial tray to the outer edges of the tibial tray to achieve the gradient decrease in porosity matching the gradient porosity of the tibia.

In embodiments, the gradient porous structure 200 of the bone contacting surface of the talus component of the total replacement implant, i.e., the bottom surface of the talus component, has a porosity that mimics the porosity of the talus. For example, the porosity of the gradient porous structure 200 of the bottom side of the talus component, contacting the talus, has a porosity between about 80% to about 90% at the center, and a porosity of about 45% to about 55% around the edges. According to preferred embodiments, the porosity at the center of talus component is about 82%, and the porosity around the edges of the tibial tray is about 52%. The porosity may decrease linearly from the center of the talus component to the outer edges of the talus component to achieve the gradient decrease in porosity matching the gradient porosity of the talus.

In embodiments, the pattern of the gradient porosity structure 200 is based on a triply period minimal surface (TPMS) function. The TPMS function may optimize the pattern of the gradient porous structure to maximize the mechanical strength of the bone contacting surface, by maximizing the coefficient of friction thereof. According to preferred embodiments, the pattern 202 of the gradient porous structure 200 is a Type P pattern, as shown in FIGS. 5A-5B. However, it would be understood that other patterns of gradient porous structures 200 designed according to a TPMS function may be suitable for use with the total replacement implants described herein, so long as the gradient porous structure 200 has a mechanical strength capable of withstanding the stress placed on a total ankle replacement implant once implanted into a patient.

According to a preferred embodiment, the total ankle replacement implant 100, as shown and described with respect to FIG. 1, has one or more bone contacting surfaces, each having a gradient porous structure 200 with the pattern 202 shown and described with respect to FIGS. 5A-5B. In some embodiments, the outer surface 120 of the stem 102 may have a gradient porous structure 200, where a porosity of the pattern (i.e., size of the pores) varies from a porosity between about 80% to about 90% at the most narrow point (i.e., the proximal end 112) of the stem 102, and a porosity of between about 60% to about 70%, such as about 65%, at the widest point (i.e., the distal end 114) of the stem 102. In some embodiments, the top surface 122 of the tibial tray and/or side surfaces 126, 128 may have a gradient porous structure 200, where the porosity of the pattern 202 varies across the surfaces. For example, the porosity of the pattern 202 may be the highest (i.e., the pores 204 are the largest) towards the center of the top surface 122 surrounding the projection 130, and may decrease to be the lowest (i.e., the pores 204 are the smallest) along the perimeter of the top surface 122. In embodiments where the side surfaces 126, 128 have a gradient porous structure 200, the porosity of the pattern 202 may be the same as the porosity along the perimeter of the top surface 122. In some embodiments, the bottom surface 150 of the talar component 108 has a gradient porous structure 200, where the porosity of the pattern 202 is highest at the center (not shown) and decrease towards the outer edges of the surface 150. The variable porosity of the gradient porous structure 200 may be similar to the density of a patient's natural bone to which the bone contacting surface of the implant 100 component is attached.

### EXAMPLES

### Example 1

### Analysis of TPMS Structures

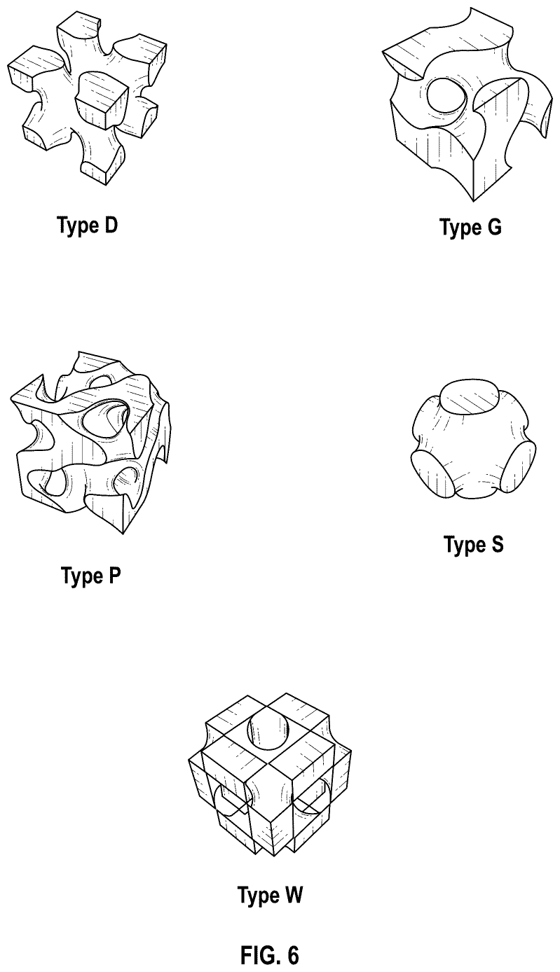

Several triply periodic minimal surface (TPMS) structures were assessed to determine which TPMS structure is best suited for use in total ankle replacement implants, such as those described herein. Two studies were conducted to optimize the microstructure (i.e., TPMS) of the porous structure, assessing both the frictional behavior and the mechanical performance of the different TPMS structures, which are shown in FIG. 6.

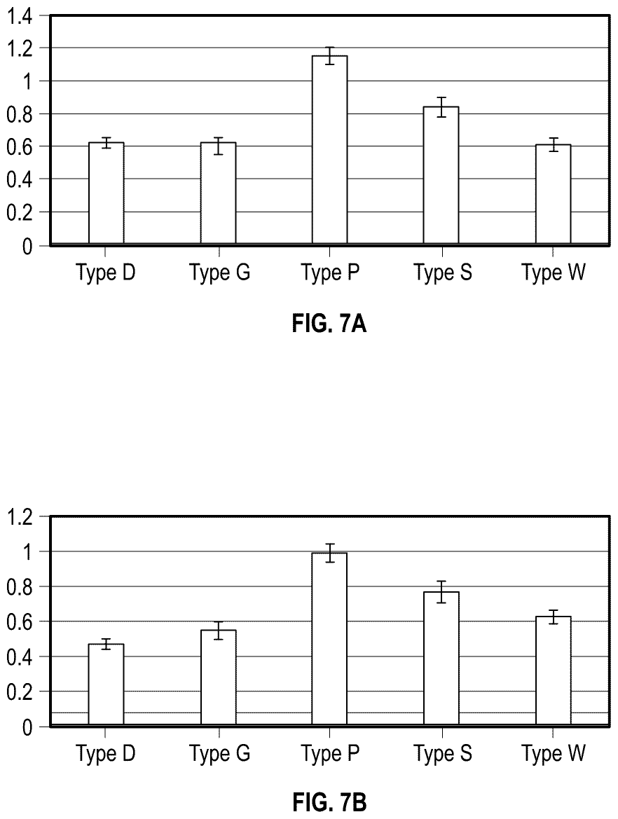

To assess frictional behavior, the five TPMS structures of FIG. 6 were tested for their static and kinetic coefficients of friction. The results of the static coefficient of friction testing is shown in FIG. 7A, and the results of the kinetic coefficient of friction testing is shown in FIG. 7B. From these tests, the Type P and the Type S structures had the highest coefficients of friction, with only the Type P structure having a static and kinetic coefficient of friction of 1 or greater, demonstrating that the Type P structure is more likely to result in a stable implant.

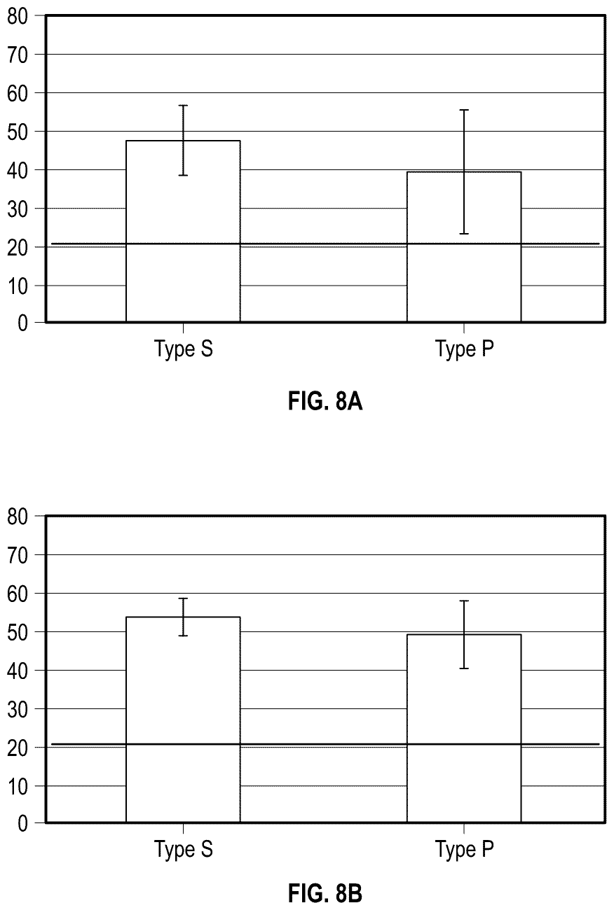

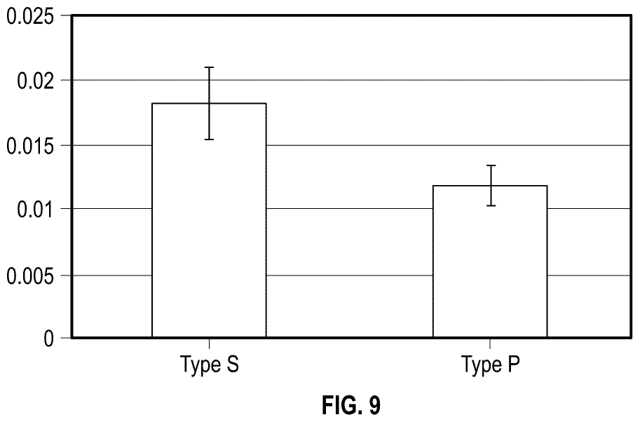

The Type P and Type S structures, having the highest coefficients of friction, were further assessed for their mechanical strengths and abrasive resistance. The mechanical strength testing involved static axial pulling and static shear testing, and the results were compared to the recommended requirements of the Food and Drug Administration (FDA). The Type S structure had a slightly larger tensile strength and shear strength than the Type P structure, as shown in FIGS. 8A and 8B, respectively. The Type S structure was similarly more abrasion resistant than the Type P structure, as shown in FIG. 9.

While the disclosure has been described with reference to a number of exemplary embodiments, it would be understood by those skilled in the art that the disclosure is not limited to such embodiments. Rather, the disclosed embodiments can be modified to incorporate any number of variations, alterations, substitutions, or equivalent arrangements not described herein, but which are commensurate with the spirit and scope of the disclosure.

That which is claimed is:

1. A total ankle replacement implant comprising:
a bone contacting surface,
wherein the bone contacting surface comprises a gradient porous structure to facilitate bone ingrowth within the bone contacting surface, and
wherein the gradient porous structure comprises a porous pattern.

2. The orthopedic implant of claim 1, wherein the porous pattern is uniform across the bone contacting surface.

3. The orthopedic implant of claim 1, wherein the porous pattern has a variable porosity across the bone contacting surface.

4. The orthopedic implant of claim 1, wherein the porous pattern has a first porosity at a center of the bone contacting surface and a second porosity at a perimeter of the bone contacting surface, wherein the first porosity is greater than the second porosity.

5. The orthopedic implant of claim 4, wherein a porosity of the porous pattern linearly decreases from the first porosity at the center of the bone contacting surface to the second porosity at the perimeter of the bone contacting surface.

6. The orthopedic implant of claim 5, wherein the first porosity is from about 80% to about 90%, preferably about 82%.

7. The orthopedic implant of claim 5, wherein the second porosity is from about 40% to about 55%, preferably about 52%.

8. The orthopedic implant of claim 1, wherein a pore size of the porous pattern is from about 200 μm to about 600 μm.

9. The orthopedic implant of claim 1, wherein an average pore size of the porous pattern is about 450 μm.

10. The orthopedic implant of claim 1, wherein the porous pattern is formed according to a triply periodic minimal surface (TPMS) function.

11. A total ankle replacement implant comprising:
a stem;
a tibial tray; and
a talus component,
wherein each of the stem, the tibial tray, and the talus component comprise at least one bone contacting surface comprising a gradient porous structure to facilitate bone ingrowth within the at least one bone contacting surface, and
wherein the gradient porous structure comprises a porous pattern.

12. The total ankle replacement implant of claim 11, wherein the porous pattern is formed according to a triply periodic minimal surface (TPMS) function.

13. The total ankle replacement implant of claim 11, wherein the porous pattern has a first porosity at a center of each bone contacting surface and a second porosity at a perimeter of each bone contacting surface, wherein the first porosity is greater than the second porosity.

14. The total ankle replacement implant of claim 13, wherein a porosity of the porous pattern linearly decreases from the first porosity at the center of each bone contacting surface to the second porosity at the perimeter of each bone contacting surface.

15. The total ankle replacement implant of claim 14, wherein the first porosity is from about 80% to about 90%, preferably about 82%.

16. The total ankle replacement implant of claim 14, wherein the second porosity is from about 40% to about 55%, preferably about 52%.

17. The total ankle replacement implant of claim 11, wherein a pore size of the porous pattern is from about 200 μm to about 600 μm, and wherein an average pore size of the porous pattern is about 450 μm.

18. The total ankle replacement implant of claim 11, wherein the gradient porous structure of the one or more bone contacting surfaces of each of the stem, the tibial tray, and the talus component is 3D printed titanium.

19. A method for inserting a total ankle replacement implant in an ankle joint of a patient, the method comprising:
resecting a tibia or a talus within the ankle joint of the patient; and
inserting a component of the total ankle replacement implant into the ankle joint of the patient, the component of the total ankle replacement implant being configured to be attached to the tibia or the talus after resection,
wherein the component of the total ankle replacement implant comprises a bone contacting surface comprising a gradient porous structure to facilitate bone ingrowth within the bone contacting surface and the tibia or the talus; and
wherein the gradient porous structure comprises a porous pattern having a first porosity at a center of the bone contacting surface and a second porosity at a perimeter of the bone contacting surface, where the first porosity is greater than the second porosity.

20. The method of claim 19, wherein the first porosity is from about 80% to about 90%, preferably about 82%, and wherein the second porosity is from about 40% to about 55%, preferably about 52%.
